(19) United States
(12) Patent Application Publication
(10) Pub. No.: US 2009/0186341 A1
(43) Pub. Date: Jul. 23, 2009

(54) Receptacle for the Separation of Tumor Cells

(75) Inventor: Michael W. Dahm, Munich (DE)

Correspondence Address: LERNER, DAVID, LITTENBERG,;KRUMHOLZ & MENTLIK, 600 SOUTH AVENUE WEST, WESTFIELD, NJ, 07090, US

(73) Assignee: HEXAL AKTIENGESELLSCHAFT, Holzkirchen (DE)

(21) Appl. No.: 11/816,742

(22) PCT Filed: Feb. 20, 2006
(86) PCT No.: PCT/EP06/01496

§ 371 Date: Dec. 23, 2008

(30) Foreign Application Priority Data
Feb. 21, 2005 (EP) 05003640.9

(51) Int. Cl. C12Q 1/68; B01L 3/14; C12Q 1/04

(52) U.S. Cl. 435/6; 422/102; 435/34

(57) ABSTRACT

The invention describes a container (1) for separating tumour cells, in particular disseminated tumour cells (9), from a biological sample, with a closed end and an end which can be opened (2, 3). It contains a thixotropic substance (4) with a specific density selected from a range with a lower limit of 1.055 g/cm3, preferably 1.057 g/cm3, in particular 1.060 g/cm3, and an upper limit of 1.070 g/cm3, preferably 1.069 g/cm3, in particular 1.065 g/cm3, and optionally a separation medium (5) in the form of a density gradient with a specific density selected from a range with a lower limit of 1.060 g/cm3, preferably 1.065 g/cm3, in particular 1.070 g/cm3, and an upper limit of 1.085 g/cm3, preferably 1.080 g/cm3, in particular 1.075 g/cm3.

The invention relates to a container for separating tumour cells from a biological sample, in particular disseminated tumour cells, with a closed end and an end which can be opened, a method of identifying tumour cells, in particular disseminated tumour cells, an arrangement of compartments of differing specific density of a biological sample and at least one separating medium for separating tumour cells, in particular disseminated tumour cells, in a container with a closed end and an end which can be opened, and a test kit.

Virtually all solid, malignant tumours have the potential to develop metastases. The process by which metastasis occurs involves the dissemination of malignant cells to remote organs, usually through the blood and lymph glands, and the development of autonomous secondary tumours. The extent of secondary tumour growth determines the prognosis of a tumour-related illness.

The benefit of tumour precautionary or post-care programmes lies in the early detection of primary tumours or the return of tumours or metastases even before they become clinically evident. Until now, it has not been possible to achieve this objective satisfactorily with the techniques based on available apparatus.

If disseminated tumour cells are detected early, e.g. in peripheral blood before a clinically identifiable tumour or metastasis occurs, a curative immunity modulation or poly-chemotherapy can be initiated. The quantification of tumour cells, in particular before and after an adjuvant therapy, therefore constitutes an important control instrument.

In addition to finding evidence of and quantifying tumour cells in body fluids, it may be of interest to carry out cytological characterisations of these tumour cells under the microscope. Under sterile conditions, isolated tumour cells can be placed in culture and corresponding cell strains established from them. Cell strains originating from disseminated, circulating tumour cells rather than from the primary tumour offer the possibility of being able to investigate process of metastasis in different ways. These cell strains may also be used for developing new and more effective tumour therapies and may help in the search for new therapeutic and diagnostic targets.

Another interesting option is to obtain the disseminated tumour cells of a cancer patient prior to treating the tumour and testing them individually to see how they respond to the tumour therapy.

Since body fluids generally contain a number of different cells, before quantifying specific cell types such as tumour cells, in particular disseminated tumour cells, it is desirable to increase their number and simultaneously eliminate as large as possible a quantity of non-tumour cells in order to facilitate quantification.

Already in the 1960s and 1970s, tumour cells were separated from haematopoetic cells on the basis of their different density. According to this data, tumour cells have a specific density of ≦1.040 to >1.065 g/cm³, whereas erythrocytes and polymorpho-nuclear leukocytes have a higher density. Lymphocytes, on the other hand, have a specific density in the range of 1.060 to 1.075 g/cm³ and thus overlap with the specific density of tumour cells.

These days, different methods are already being used to isolate and characterise disseminated tumour cells. One group of methods, such as filtration and density gradient centrifugation for example, is based on the physical properties, such as the size or density of the cells.

Another group of methods uses the specific immunological properties of tumours or blood cells, whereby the cells are bonded by means of specific antibodies and either enriched by positive selection or depleted by negative selection. If using positive selection to increase the number of epithelial tumour cells, the cells are marked with specific antibodies to epithelial cell-specific antigens, such as EPCAM (Epithelial Cell Adhesion Molecule, in other words HEA or 17-1A antigen) and cytokeratine, and coupled on magnetic particles or fluorescent molecules. Marked in this manner, the tumour cells are then enriched by means of a cell separator, such as MACS (Magnetic Cell Sorting) or FACS (Fluorescence Activated Cell Sorting). The disadvantage of positive selection is that only tumour cells of epithelial origin can be detected.

These selection methods are very complex and time-consuming. The cells are subjected to a high degree of stress due to the nature of the method. To an extent, this can bring about a detrimental change to the cell morphology so that effective cytological evaluation is no longer possible. Furthermore, the enriched cells can no longer be placed in culture and expanded.

In addition, the individual antigen manifestations on and in the blood or tumour cells can be individually regulated in different ways. In the case of a low antigen manifestation on the cell surface, this can lead to poor separation results. Blood or tumour cells can also be expressed with a view to selecting undesired surface antigens. This can lead to non-specific enrichment or depletion and to falsely positive or falsely negative results. The antibodies based on selection and identification methods depend to a large extent on the quality of whatever antibodies are used.

Viewed overall, cellular, in particular immunocytological identification of the cells is regarded as highly specific but is not very sensitive. In both cases, however, these methods are complex and expensive and therefore barely suitable for routine testing.

Molecular diagnosis, in particular amplification methods such as PCR (Polymerase Chain Reaction), RT-PCR (Reverse Transcription Polymerase Chain Reaction) and QPCR (Quantitative Reverse Transcription Polymerase Chain Reaction) used for detecting nucleic acids specific to tumour cells, such as DNA or RNA, offers an interesting alternative to the cellular identification methods. All of the methods based on PCR technology (Polymerase Chain Reaction) are highly sensitive but are dependent to a large degree on the quality of the nucleic acids in a sample. Once the blood sample has been taken, the cellular nucleic acids, in particular RNA, can deteriorate very rapidly due to intra- and extra-cellular enzymes, so that there are not enough nucleic acids specific to cells, in particular tumour cells, or these can no longer be quantified or detected at all. In addition, it has been proven that cells and in particular tumour cells can reduce cell-specific and tumour cell-specific gene expression profiles after taking the blood sample. These changes in the gene expression profile is all the more sustained, the longer the tumour cells remain in the unprocessed blood sample. Since the subsequent nucleic acid purification process, in particular the RNA extraction, concentration and re-expression processes, are very susceptible to contamination with exogenic nucleases, in particular with RNases, falsely negative results can be produced in spite of the fact that the molecular diagnosis is not overly sensitive.

A system known from the prior art for enriching tumour cells is the OncoQuick® System. The circulating tumour cells are enriched with OncoQuick® on the basis of a density gradient which is specially adapted to the density of the cells to be separated and which is optimised for 15 to 30 ml of full blood and bone marrow. OncoQuick® comprises a centrifuging tube, which is divided into two compartments by means of a porous separating disc. The bottom compartment contains a separating medium. The top compartment can be filled with up to 30 ml of the sample to be tested. Due to the specific properties of the separation medium and the separating disc, the unwanted blood or bone marrow cells form a sediment in the bottom compartment during centrifugation and thus force a corresponding volume of the separating medium into the top compartment. The cell fraction with the lower density, which contains the tumour cells amongst other things, is concentrated in the inter-phase in the top compartment between plasma and separating medium, from where it can be removed.

Tests undertaken using OncoQuick® have now surprisingly shown that the age of the blood is decisive in terms of the degree and quality (i) of enrichment of disseminated tumour cells and in particular (ii) as regards the removal of unwanted non-tumour cells. When processing 20 ml of peripheral blood with OncoQuick®, the degree of reduction of unwanted non-tumour cells decreases after 2 hours or after 24 hours compared with the original blood cells originally present in the blood sample, with a relative log reduction factor of ca. log 5.9 to ca. log 3.8. The reason for this is a non-specific change dependent on cell type, in particular an increase in the specific density of the blood cells.

Accordingly, the objective of the invention is to propose a method and components for implementing a method whereby tumour cells can be enriched rapidly and efficiently. A partial objective is to prevent or reduce as far as possible detrimental changes on (i) the cellular and (ii) molecular level, both with respect to the method of enrichment and the subsequent cellular or molecular identification method.

This objective is achieved by the invention, independently in each case, by means of (A) a container with a closed end and an end which can be opened, in which is placed (a) a thixotropic substance with a specific density selected from a range with a lower limit of 1.055 g/cm³, preferably 1.057 g/cm³, in particular 1.060 g/cm³, and an upper limit of 1.070 g/cm³, preferably 1.069 g/cm³, in particular 1.065 g/cm³, and optionally (b) a separation medium in the form of a density gradient with a specific density selected from a range with a lower limit of 1.060 g/cm³, preferably 1.065 g/cm³, in particular 1.070 g/cm³, and an upper limit of 1.085 g/cm³, preferably 1.080 g/cm³, in particular 1.075 g/cm³, (B) a method comprising the steps of a) providing a container, b) placing the biological samples in the container, c) centrifuging the container in order to separate the biological samples into at least a bottom and top compartment, d) conducting a molecular-biological, immunological and/or cellular test on the tumour cells from the biological sample disposed in a compartment above the thixotropic substance and/or porous barrier after centrifugation, (C) an arrangement of compartments of differing specific density, whereby initially, (a) a separation medium in the form of a density gradient with a specific density selected from a range with a lower limit of 1.06 g/cm³, preferably 1.065 g/cm³, in particular 1.070 g/cm³, and an upper limit of 1.085 g/cm³, preferably 1.080 g/cm³, in particular 1.075 g/cm³, and then optionally (b) a thixotropic substance with a specific density selected from a range with a lower limit of 1.055 g/cm³, preferably 1.057 g/cm³, in particular 1.060 g/cm³, and an upper limit of 1.070 g/cm³, preferably 1.069 g/cm³, in particular 1.065 g/cm³ are originally placed in the region of the end which can be opened, and, in the region of the closed end, (c) a space is provided with a volume sufficient to accommodate the biological sample, and after the centrifugation process, there are, starting with (i) the compartment in the region of the bottom end of the container, erythrocytes, and then (ii) a compartment of leukocytes, monocytes, lymphocytes and optionally (iii) some of the separation medium, in turn followed by (iv) the thixotropic substance, and then (v) a compartment of diluted separation medium, followed by (vi) a compartment of plasma with thrombocytes and tumour cells, in particular disseminated tumour cells, and optionally (vii) a space, (D) a test kit appropriate for this purpose and (E) the use thereof.

It has also advantageously been found that optimum separation properties of the biological sample can be achieved if the disseminated tumour cells can be separated as quickly as possible after taking the sample. It is of particular advantage if the process of taking the sample and separating out the disseminated tumour cells contained in the sample can be implemented in the same container.

This being the case, in a preferred embodiment of the invention, a container which can be evacuated is provided as the system for taking the blood sample so that the sample can be taken automatically due to an already existing vacuum, thereby obviating the need for the person taking the sample to come into contact with the biological sample.

In another preferred embodiment of the invention, this container can be centrifuged and the container used to obtain the biological sample, and, if the latter is a body fluid which can be obtained by puncturing, can be centrifuged directly without the need for an intermediate step in order to transfer the biological sample from the vessel used to take the sample into the vessel used for centrifugation.

It has also proved to be of advantage if anti-coagulant and/or aggregation-inhibiting substances are used, which on the one hand prevents the aggregation of thrombocytes on tumour cells and/or the body fluid is freed of substances which promote aggregation of thrombocytes on tumour cells and, on the other hand, prevents coagulation of the biological sample.

The thixotropic substance is a material selected from a group comprising silicic acid, bentonite, hectorite, kaolin, alginate and/or a mixture thereof, and the substance is liquefied by the action of mechanical forces and solidified again when the mechanical forces are removed.

The separation medium is an aqueous solution of at least one polymer, in particular saccharide and/or diatrizoate, such as Percoll®, Ficoll® or media with the same separation properties, thereby producing a density gradient by means of which a simple and rapid centrifugation procedure can be run in order to separate cells on the basis of their different density.

In another embodiment, the separation medium and/or the thixotropic substance contains or contain one or more dyes, thereby enabling a clear boundary to be made visible between the separation medium respectively thixotropic substance and the tumour cells of the biological sample.

In another embodiment, a porous barrier is provided in the form of a membrane, flap, frit, sieve and/or filter so that the container is divided into a top and bottom compartment, in which case the cell separation medium is disposed in the bottom compartment and the body fluid is introduced into the top compartment. In this respect, it has proved to be of advantage when introducing the biological sample if no mixing takes place with the cell separation medium. Any mixing of the compartments is also prevented after centrifugation.

The preferred embodiment is a container 1 which can be evacuated and is sub-divided into two compartments, a bottom 8 and a top 7, by means of a thixotropic gel 4 disposed in the bottom third of the container. The purpose of the separation medium 5 placed underneath the thixotropic gel is (i) to hold the thixotropic gel in position and (ii) to ensure that the heavier cells are able to migrate through the thixotropic gel (or liquefied gel) unobstructed during centrifugation and can accumulate in the space originally formed by the separation medium. Introducing a porous barrier 5 (membrane, flap, frit, sieve and/or filter) in addition offers the following possibilities:

### EMBODIMENT 1

no additional device because a vacuum can be created.

### EMBODIMENT 2

a thixotropic substance additionally seals the porous barrier (membrane, frit, sieve and/or filter, flap) thereby making it easier to create the vacuum. In this case, the thixotropic substance has a lower density than the separation medium and the separation medium assumes the separation function of the thixotropic gel. The porous barrier (membrane, frit, sieve and/or filter, flap) is also used in addition to the thixotropic substance without separation medium. In this case, the barrier assumes the function of the separation medium and lies underneath the thixotropic substance. The purpose of the porous barrier disposed underneath the thixotropic gel is (i) to hold the thixotropic gel (i) in position and (ii) to guarantee that the heavier cells are able to migrate through the thixotropic gel (or liquefied gel) unobstructed during centrifugation and can accumulate in the space originally formed by the porous barrier.

The porous barrier (membrane, flap, frit, sieve and/or filter) is used in addition to the thixotropic substance and separation medium.

The porous barrier has a thickness selected from a range with an upper limit of 15 mm, preferably 10 mm, in particular 5 mm, and a lower limit of 0.1 mm, preferably 1 mm, in particular 2 mm, thereby providing sufficient strength to withstand the centrifugation forces undamaged.

Porous barriers with a pore size selected from a range with an upper limit of 150 μm, preferably 100 μm, in particular 50 μm, and a lower limit of 1 μm, preferably 10 μm, in particular 20 μm, ensure that during centrifugation, liquids as well as corpuscular elements which have a higher density than the separation medium used are able to pass through the barrier unhindered and the separation medium is therefore forced into the top compartment during centrifugation. The tumour cells move so that they lie at a level above the porous barrier.

In another embodiment, the porous barrier is made from a hydrophobic material and/or is provided with a hydrophobic coating, thereby preventing cells or other particulate elements from sticking to it.

It has also proved to be of advantage if the porous barrier is bounded by an elastomer, so that a tight and in particular liquid-tight closure can be produced between the internal wall of the container and the barrier itself.

By providing a closure element which can be inserted, a passage can be made available whilst the centrifugal forces are active, which can then be easily closed again.

A projection may be provided on the internal face, which holds the porous barrier in its end position, thereby forming a bottom compartment, because the barrier is blocked in the desired position during and after taking the blood sample as well as during and after centrifugation.

As a result of the method proposed by the invention, a number of different biological samples may be used, such as a body fluid from a group comprising blood and bone marrow, urine, saliva, lymph, exudate, transudate, spinal fluid, semen, or dispersed tissue and/or fluids from natural or non-natural body cavities, irrespective of the origin of the biological sample. This obviates the need for any adaptation steps for samples of different origins.

It is possible to identify tumour cells of metastasing, in particular micro-metastasing tumours and/or neoplasms from a group including (1) solid tumours, comprising (i) epithelial tumours, such as lung carcinomas (lung carcinomas with small cells and not-small cells), gastrointestinal tumours (liver cell carcinoma, pancreatic carcinoma, oesophageal carcinoma, stomach cancer, intestinal cancer, colon-rectal carcinoma), breast cancer, kidney and suprarenal tumours, cancer of the bladder and prostate carcinoma, and (ii) non-epithelial tumours, such as melanoma, neuroblastomas, brain tumours, rhabdomyosarcoma, leiomyosarcoma or teratocarcinoma, and (2) haematological tumours, such as for example T-cell lymphoblastomas, T-cell leukaemia, chronic myeloid leukaemia, acute lymphatic leukaemia, chronic lymphatic leukaemia, and/or lymphoma, thereby enabling the same method to be used to identify a plurality of different tumour-related illnesses.

The biological sample may be diluted, which will result in better separation properties, for example if the biological sample is disposed in a small volume, the volume is increased and/or the viscosity of the biological sample is reduced.

It has also proved to be of advantage if the blood is taken in a coagulation-inhibiting substance, thereby preventing the blood from coagulating during the process of separating the tumour cells.

It has also proved to be of advantage if the biological sample has at least one aggregation-inhibiting substance added to it, thereby preventing any aggregation of thrombocytes on tumour cells and ensuring that the biological sample is free of substances which promote an aggregation of thrombocytes on tumour cells.

Centrifugation takes place with a g-number selected from a range with a lower limit of 500 g, preferably 800 g, in particular 1000 g and an upper limit of 2500 g, preferably 2000 g, in particular 1500 g, thereby resulting in optimum separation properties of the disseminated tumour cells from the rest of the constituents of the biological sample. Centrifugation also advantageously takes place without acceleration and without using a brake.

The centrifugation lasts for a period with an upper limit of 60 min, preferably 45 min, in particular 30 min, and a lower limit of 5 min, preferably 10 min, in particular 20 min, thereby making separation of the tumour cells as efficient as possible and making the subsequent process of obtaining the tumour cells from the top compartment easier.

Centrifugation preferably takes place at 4° C., and the preferred densities of (i) the thixotropic substance and optionally (ii) the separation medium are adjusted to suit this temperature. In the case of centrifugation at 20° C., the specific density of (i) the thixotropic substance and optionally (ii) the separation medium must be increased accordingly.

After centrifugation and prior to removing the compartment containing the enriched tumour cells, the container may be cooled, thereby resulting in a clearer demarcation between the compartment containing no tumour cells and the adjacent compartment containing the tumour cells.

The tumour cells may be obtained from a compartment in and/or underneath the plasma compartment, and the disposition of the tumour cells in or adjacent to the plasma compartment will produce an optimum environment for the tumour cells. This environment on the one hand causes the cells to be preserved in terms of their morphology and on the other hand prevents the cells from being broken down by various enzymes so that their contents to be tested subsequently, such as DNA or RNA, are not attacked by DNAses or RNases.

The disseminated tumour cells may be removed from the top compartment manually, semi-automatically or automatically, in which case a plurality of different parameters of the top compartment next to the disseminated tumour cells can be determined, for example plasma can be automatically obtained by means of a laboratory robot and serological routine parameters can be tested on the removed plasma.

For the subsequent diagnosis, a method may be used from a group comprising immunocyto-chemical dying, PCR (Polymerase Chain Reaction), RT-PCR (Reverse Transcriptase-Polymerase Chain Reaction), QPCR (Quantitative Reverse Transcriptase-Polymerase Chain Reaction), cell culture, FISH (Fluorescence in-situ Hybridisation) and/or FACS (Fluorescence activated cell sorter), in which case it is possible to run both molecular biological, immunological and/or cellular tests as required. Different identification methods may be used to obtain a result which is confirmed by means of a selected method. A conclusion about the presence of a tumour is of vital importance to a patient and it is therefore of enormous relevance that no falsely positive results can occur.

In addition to the container proposed by the invention, the test kit may comprise a vessel with a washing buffer, optionally in concentrated format, which means that the laboratory or institute conducting the test will need no additional reagents.

The kit may also contain other sample vessels, to which the washed tumour cells are transferred and in which other method steps and tests are conducted.

The invention will be described in more detail below in the explanations given below.

The invention will be explained in more detail below with reference to an example of an embodiment illustrated in the appended drawing.

The drawing comprises:

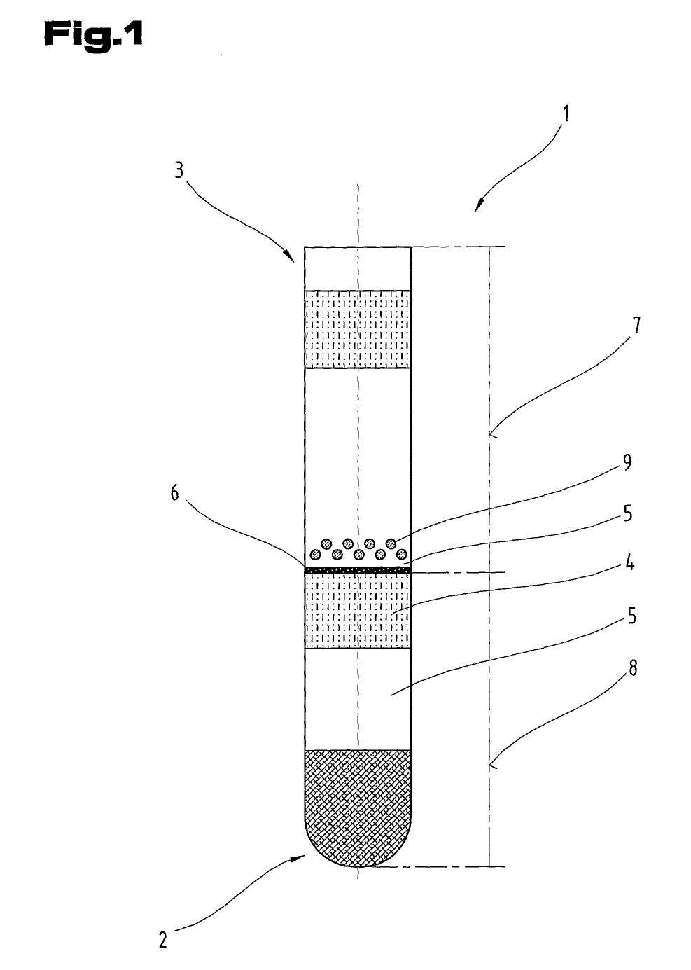

FIG. 1 illustrating a longitudinal section of the disposition of the compartments 7, 8 in the container 1.

Firstly, it should be pointed out that the same parts described in the different embodiments are denoted by the same reference numbers and the same component names and the disclosures made throughout the description can be transposed in terms of meaning to same parts bearing the same reference numbers or same component names. Furthermore, the positions chosen for the purposes of the description, such as top, bottom, side, etc,. relate to the drawing specifically being described and can be transposed in terms of meaning to a new position when another position is being described. Individual features or combinations of features from the different embodiments illustrated and described may be construed as independent inventive solutions or solutions proposed by the invention in their own right.

This invention relates to a method of enriching or depleting tumour cells from a biological sample, whereby one or more substances is/are added to the biological sample in a fluid in a container 1 and centrifuged.

The method proposed by the invention may be used for both enriching and depleting tumour cells, in particular disseminated tumour cells 9, depending on which compartment is subjected to additional processing after centrifugation. Accordingly, no distinction will be made between these two possible treatments and instead, the description will refer generally to enrichment of tumour cells, even though both options are covered by the invention.

The biological sample may be both a body fluid or may originate from an organic tissue. The sample may be of both human and animal origin. The body fluid may be blood, urine, saliva, lymph, exudate, transudate, spinal fluid, semen, fluids from natural or non-natural body cavities or, if using tissue, may be bone marrow tissue or any other dispersed and homogenised tissue.

The container 1 for taking the biological sample and into which the biological sample is introduced has a closed end 2 and an end 3 which can be opened. Contained in the container 1 is a thixotropic substance 4 with a specific density selected from a range with a lower limit of 1.055 g/cm³, preferably 1.057 g/cm³, in particular 1.060 g/cm³ and an upper limit of 1.070 g/cm³, preferably 1.069 g/cm³, in particular 1.065 g/cm³.

In one embodiment, the container 1 may additionally contain a separation medium 5 in the form of a density gradient with a specific density which is the same as or higher than that of the thixotropic substance 4, selected from a range with a lower limit of 1.060 g/cm³, preferably 1.065 g/cm³, in particular 1.070 g/cm³ and an upper limit of 1.085 g/cm³, preferably 1.080 g/cm³, in particular 1.075 g/cm³.

In an alternative embodiment, the separation medium 5 may also have a specific density selected from a range with a lower limit of 1.055 g/cm³, preferably 1.057 g/cm³, in particular 1.060 g/cm³ and an upper limit of 1.070 g/cm³, preferably 1.068 g/cm³, in particular 1.065 g/cm³ and a porous barrier 6.

In the simplest embodiment, the container contains only a thixotropic substance 4, in particular a thixotropic gel, with a specific density of 1.057 to 1.069 g/cm³, in particular 1.060 g/cm³. The thixotropic substance 4 is disposed in the region of the closed end of the container 1.

In a variant of the simplest embodiment, there is provided in addition to the thixotropic substance 4 a separation medium 5 in the form of a density gradient with a specific density selected from a range of 1.060 to 1.085 g/cm³, in particular 1.065 to 1.070 g/cm³. In this embodiment, the separation medium 5 is disposed in the region of the closed end 2 and the thixotropic substance 4 is contained above the separation medium 5 in the container 1.

In an alternative embodiment of the container 1, the thixotropic substance 4 and the separation medium 5 may be of approximately the same density selected from a range of 1.055 g/cm³ to 1.070 g/cm³, in which case a porous barrier 6 is provided in addition. In this embodiment, the separation medium 5 is disposed in the region of the closed end 2 of the container 1 with the porous barrier 6 lying above and then the thixotropic substance 4 lies in the direction towards the end 3 of the container 1 which can be opened.

The separation medium holds the thixotropic substance 4 in position before and whilst taking the sample as well as during and after centrifugation in the container 1. The separation medium 5 also ensures that the denser components of the body fluid to be tested, preferably peripheral, venous, arterial blood, or a mixture thereof such as blood from the finger tip, central venous, central arterial and cordal blood such as bone marrow, in particular erythrocytes, monocytes, granulocytes and some of the denser lymphocytes, are able to migrate through the thixotropic substance 4 unhindered and reach the space formed by the separation medium 5.

As this happens, the corresponding volume of separation medium 5 is forced, causing the thixotropic substance 4 lying above to be pushed upwards and/or the separation medium 5 is pre-diluted by the fluid displaced with the downwardly migrating cells so that a specific quantity of the diluted separation medium 5 moves so that it lies above the thixotropic substance 4 after centrifugation.

The thixotropic substance 4 may be silicic acid, bentonite, hectorite, kaolin, alginate and a mixture thereof. The thixotropic substance 4 may be provided in gel format and enables the container 1 to be separated into at least one bottom and at least one top compartment 7, 8, and, because of the specific density, enrichment of the disseminated tumour cells 9 will take place in the top compartment 7 and the simultaneous removal of undesired cells will take place in the bottom compartment 8. The thixotropic substance 4 also enables the top compartment 7 to be evacuated.

The separation medium 5, which is present in the form of a density gradient, is provided in the form of an aqueous solution of at least one polymer, in particular saccharide or diatrizoate, known under the trade names of Percoll® or Ficoll® or a substance similar to Percoll or Ficoll. In the case of containers 1 made from plastic, it is preferable to use Percoll® and for containers 1 made from glass, it is preferable to use Ficoll®.

In another embodiment of the container 1 proposed by the invention, a quantity of dye may be admixed with the separation medium 5 and/or the thixotropic substance 4. The dye added to the separation medium 5 or to the thixotropic substance 4 may be Trypan blue. This addition makes it easier to see the boundary between the individual phases and compartments of differing density.

At its open end, the container 1 can also be closed. The container 1 may also be evacuated. The container 1 proposed by the invention may be evacuated in a manner similar to that of an evacuated blood sample tube known from the prior art and may contain a thixotropic substance 4 and a separation medium 5 and optionally a porous barrier 6.

If blood is used as the biological sample, for example, it may be taken directly using the evacuated container 1 proposed by the invention and the tumour cells 9 are separated after centrifuging the container 1.

In order to use the evacuated container 1 to separate disseminated tumour cells 9 from the rest of the blood constituents, the container 1 can be centrifuged.

The biological sample need not necessarily be taken using the container 1 directly and instead it may be transferred to the container 1 after being obtained by some other means. If tissue obtained from a biopsy is used, for example, a fluid may be added, e.g. a buffer, and the sample is dispersed and homogenised. In this case, the biological sample is not transferred to the container 1 until after the sample has been obtained.

Depending on the nature of the body fluid or tissue, it is either diluted beforehand with a diluent, preferably a buffer, or introduced undiluted directly into the container 1.

If blood is used as the biological sample, anti-coagulant and/or aggregation-inhibiting substances are also introduced into the container 1.

In order to prevent agglutination of the blood, agglutination-inhibiting substances may be added, such as EDTA or citrate or heparin or CPD (citrate, phosphate, dextrose) or similar substances, for example.

Aggregation-inhibiting substances may be added to the buffer used as a diluent, for example. The preferred buffer is Dulbecco PBS (phosphate buffered saline). Substances which are suitable for preventing unwanted aggregation of thrombocytes on tumour cells include EDTA, citrate and ACD-A (Acid Citrate Dextrose), for example. In addition or instead, substances which promote aggregation of thrombocytes on tumour cells may be removed from the body fluids. These include ions, such as magnesium and calcium ions, for example.

The thickness of the porous barrier 6 is selected from a range with a top limit of 15 mm, preferably 10 mm, in particular 5 mm, and a lower limit of 0.1 mm, preferably 1 mm, in particular 2 mm.

The pore size of the porous barrier 6 is selected from a range with an upper limit of 150 μm, preferably 100 μm, in particular 50 μm, and a lower limit of 1 μm, preferably 10 μm, in particular 20 μm.

The porous barrier 6 may also be made from a hydrophobic material and/or may be provided with a hydrophobic coating.

In one embodiment of the porous barrier 6, an elastomer is provided or an elastomer bounds the porous barrier.

In an alternative embodiment, the porous barrier 6 may also be provided in the form of a plunger, by means of which the biological sample is drawn into the container 1 by negative pressure. This plunger may be provided with a porous barrier 6 and surrounded with an elastomer, such as rubber for example, so that the plunger closes and seals the container 1. If the container 1 is provided with a displaceable porous barrier 6 in the form of a plunger, the thixotropic substance 4 assumes the function of covering the side of the porous barrier 6 facing the open end of the container 1 whilst the blood sample is being taken so that a vacuum is created in the container 1. In this embodiment, the cells are separated by the previously introduced separation medium 5.

In another embodiment, a ring, pin, projection, etc., may be provided in the region of the closed end 2 of the container 1 which blocks the plunger in a desired position during and after taking the blood sample, thereby forming a bottom compartment. The separation medium 5 is placed in this bottom compartment 1 beforehand. The density of the separation medium 5 in this embodiment is selected from a range of 1.055 g/cm³ to 1.070 g/cm³, in particular 1.057 g/cm³ to 1.063 g/cm³ and is more particularly preferably 1.060 g/cm³. Due to subsequent centrifugation, disseminated tumour cells 9 are concentrated in the top compartment and undesired blood cells are removed into the bottom compartment.

In another embodiment of the porous barrier 6, it is possible to provide a closure element which can be inserted.

As described above, having provided the container 1, the biological sample is added to it based on the method proposed by the invention. The biological sample may be diluted with aggregation-inhibiting and/or coagulation-inhibiting and/or isotonic solutions. The aggregation-inhibiting and coagulation-inhibiting substances may be used in lyophilised or sprayed format on the internal face of the container 1 and in particular may be disposed inside the top compartment 7.

In another method step, the container 1 is centrifuged in order to separate the disseminated tumour cells 9 from the rest of the biological sample and is separated into at least a bottom and top compartment 7. Centrifugation takes place with a g-number selected from a range with a lower limit of 500 g, preferably 800 g, in particular 1000 g and an upper limit of 2500 g, preferably 2000 g, in particular 1500 g. The biological sample is centrifuged for a period with an upper limit of 60 min, preferably 45 min, in particular 30 min and a lower limit of 5 min, preferably 10 min, in particular 20 min.

The centrifugation preferably takes place at +4° C.; alternatively, however, it may also be run at room temperature. Centrifugation preferably takes place at 4° C., in which case the preferred densities of (i) the thixotropic substance and optionally (ii) the separation medium are adjusted for this temperature. Since density decreases with rising temperatures, if centrifugation takes place at 20° C., the specific density of (i) the thixotropic substance and optionally (ii) the separation medium must be increased accordingly.

After centrifugation and prior to removing the compartment containing the enriched tumour cells, the container 1 may additionally be cooled. Cooling, in particular brief, rapid cooling, enables the erythrocytes and leukocytes disposed in the bottom compartment 8 of the container 1 to be immobilised. Cooling may take place in liquid nitrogen. Cooling prevents any mixing of cells from different compartments, thereby ruling out falsely positive test results.

The disseminated tumour cells 9 are obtained from a compartment 7 above the thixotropic substance 4 or above the porous barrier 6. Plasma, plasma/PBS or a plasma/buffer mixture containing proteins of the plasma, lies in the uppermost compartment.

Plasma keeps cells alive during the period of transportation to the testing laboratory. After purification based on the method proposed by the invention, e.g. still at the hospital or at the premises of the authorised doctor, the cell fraction containing the enriched and disseminated tumour cells 9 is contained in such a top compartment 7, i.e. in a large volume of plasma. Plasma on the one hand offers a physiological environment and thus preserves the cell morphology and the functionality of the cells. This therefore protects the biological sample against destructive enzyme activity, for example, and in particular protects the cellular RNA against deterioration due to RNases.

The tumour cells are removed either (i) manually, i.e. the container 1 is opened and the top compartment 7 is transferred to a new vessel by decanting or pipetting, (ii) semi-automatically, for example by transferring into an evacuated sample cartridge with properties which conserve the cells and nucleic acid, such as described in patent specification WO 03/09131, or (iii) automatically, for example by a pipetting robot. Washing and preservation steps may also be carried out on the fluid of the top compartment 7. It is possible to remove the tumour cells 9 only or alternatively the entire top compartment 7.

From the top compartment 7, it is also possible to determine routine parameters relating to serology, as is standard laboratory practice.

The test is run after separating the tumour cells from the rest of the biological sample. A test may be run on a molecular-biological, immunological and/or cellular basis. The identification methods which may be used include immunocyto-chemical dying, polymerase chain reaction, reverse transcriptase-polymerase chain reaction, cell culture, fluorescence in-situ hybridisation and/or fluorescence activated cell sorting. The list of methods which may be used once the disseminated tumour cells 9 have been obtained is based purely on examples and does not claim to be complete by a long way.

The biological sample can be processed within the shortest time using the container 1 proposed by the invention and the method proposed by the invention. Optimum separation properties are obtained if the disseminated tumour cells 9 are separated at least on the same day that the samples are obtained, e.g. on the day the blood sample is taken. If the blood is older than 1 day, a higher proportion of undesired blood cells remains in the top compartment 7 after centrifugation, which also contains the enriched disseminated tumour cells 9. As a result, a higher quantity of cells and hence a higher overall quantity of nucleic acid concentrates and can thus place a higher strain on the cellular or molecular identification method. For example, more object substrate may be needed for immunocytological identification methods. To rule out overloading the reverse transcriptase-polymerase chain reaction (RT-PCR) or quantitative polymerase chain reaction (QPCR) due to higher quantities of nucleic acid, only part-quantities of the biological sample are used. This can in turn reduce the information which can be obtained from the test because only a proportion of the biological sample originally taken is used.

In the case of blood which is more than 1 day old, the likelihood of the tumour marker being enriched with non-tumour cells is also increased, which means that falsely positive results can be expected. In older biological samples, the cellular RNA also deteriorates very rapidly, thereby incurring the risk that falsely negative results will be obtained. The method proposed by the invention, on the other hand, enables the biological sample to be processed immediately by the doctor in question or at an out-patient department of a clinic, thereby offering an optimum starting point for subsequent cellular or molecular testing.

After running the centrifugation process, there are at least two compartments, a top 7 and a bottom 8 compartment. The two compartments are in turn sub-divided into compartments of differing specific density. FIG. 1 illustrates the disposition of the individual compartments. Starting with the compartment in the region of the closed end 2 of the container 1, there are (i) erythrocytes, then (ii) a compartment with leukocytes, monocytes, lymphocytes, and optionally (iii) a part of the separation medium 5, and then in turn (iv) the thixotropic substance 4, and optionally the porous barrier 6, followed by (v) a compartment of the separation medium 5 which was diluted with fluid by the cells migrating downwards, and then (vi) a compartment containing plasma with thrombocytes and at the bottom boundary of the compartment, the disseminated tumour cells 9 and optionally (vii) a space.

The container 1 may be part of a test kit. In addition to a container 1, the test kit may also contain a washing buffer, optionally in concentrated format, in solution or in powdered form, which is used in a dilution with a solvent

The test kit may also contain other sample vessels into which (i) the disseminated tumour cells 9, and in a preferred application (ii) the entire top compartment 7 containing the disseminated tumour cells 9 can be transferred after centrifugation. The requisite washing, enrichment and preservation steps performed on the enriched tumour cells 9 may be carried out in these vessels so that the cells or the nucleic acids taken from the cells can be transported for further cellular or molecular testing.

The specific densities of the compartments described above are broken down as follows, starting from the closed end 2 of the container 1:

| Compartment containing | Specific density (g/cm³) |
|---|---|
| Erythrocytes | 1.092 to 1.097 |
| Leukocytes | 1.075 to 1.085 |
| Lymphocytes/monocytes | 1.060 to 1.075 |
| Separation medium | 1.070 to 1.085 |
| Thixotropic substance | 1.055 to 1.070 |
| Separation medium* | <1.055 to <1.070 |
| Tumour cells, disseminated | 1.040 to 1.70 |
| Plasma and thrombocytes | 1.034 to 1.040 |

*the separation medium is diluted by the cells transporting fluid as they move up from the bottom.

The disseminated tumour cells 9 may be used to identify metastasing, in particular micro-metastasing tumours and/or neoplasms from a group including (1) solid tumours, comprising (i) epithelial tumours such as lung carcinoma (lung carcinoma with small cells and not-small cells), gastrointestinal tumours (liver cell carcinoma, pancreatic carcinoma, oesophagus carcinoma, stomach cancer, intestinal cancer, colon-rectal carcinoma), breast cancer, liver and suprarenal tumours, cancer of the bladder and prostate carcinoma, and (ii) non-epithelial tumours, such as melanoma, neuroblastomas, brain tumours, rhabdomyosarcoma, leiomyosarcoma or teratocarcinoma for example, and (2) haematological tumours such as T-cell lymphoblastomas, T-cell leukaemia, chronic myeloid leukaemia, acute lymphatic leukaemia, chronic lymphatic leukaemia and/or lymphomas, for example.

If thrombocytes have also been enriched in the collected tumour cell fraction, it may also be of benefit to cellular testing if the collected cells are applied to an object carrier and washed at least once with a buffer e.g. PBS, or PBS with 0.1% to 7% BSA) and separated from the cells by centrifugation at ca. 200 g for 10 min.

### EXAMPLE OF AN EMBODIMENT

A blood vessel contains a thixotropic substance 4 with a specific density of 1.060 g/cm³ and a separation medium 5, Percoll, with a specific density of 1.070 g/cm³. Using methods known from the prior art, a blood sample is taken from a vein with a needle holder and introduced into the container 1. Also contained in the container 1 is an aggregation-inhibiting, coagulation-inhibiting (heparin) substance. Centrifuging takes place with slow acceleration and without a brake for 20 min at 1000 g and 4° C. After centrifugation, the entire compartment above the thixotropic substance 4 is removed and subjected to further testing.

The drawing illustrates but one possible embodiment of the container 1, in particular the disposition of the compartments in the container, and it should he pointed out at this stage that the invention is not restricted to the particular embodiment specifically illustrated here, and instead, other individual design variants may be used in different combinations with one another and these possible variations lie within the reach of the person skilled in this technical field given the disclosed technical teaching. Accordingly, all conceivable design variants which can be obtained by combining individual details of the design variants described and illustrated are possible and fall within the scope of the invention.

For the sake of good order, finally, it should be pointed out that, in order to provide a clearer understanding of the structure of the container, it and its constituent parts are illustrated to a certain extent out of scale and/or on an enlarged scale and/or on a reduced scale.

The objective underlying the independent inventive solutions may be found in the description.

### LIST OF REFERENCE NUMBERS

1 Container

2 Closed end

3 End which can be opened

4 Thixotropic substance

5 Separation medium

6 Porous barrier

7 Top compartment

8 Bottom compartment

9 Disseminated tumour cells

1. Container (1) for separating tumour cells, in particular disseminated tumour cells (9), from a biological sample, with a closed end and an end which can be opened (2, 3), wherein it contains a thixotropic substance (4) with a specific density selected from a range with a lower limit of 1.055 g/cm³, preferably 1.057 g/cm³, in particular 1.060 g/cm³, and an upper limit of 1.070 g/cm³, preferably 1.069 g/cm³, in particular 1.065 g/cm³, and optionally a separation medium (5) in the form of a density gradient with a specific density selected from a range with a lower limit of 1.060 g/cm³, preferably 1.065 g/cm³, in particular 1.070 g/cm³, and an upper limit of 1.085 g/cm³, preferably 1.080 g/cm³, in particular 1.075 g/cm³.

2. Container (1) as claimed in claim 1, wherein the container (1) can be evacuated.

3. Container (1) as claimed in claim 1 or 2, wherein the container (1) can be centrifuged.

4. Container (1) as claimed in one of claims 1 to 3, wherein it contains at least one anti-coagulating and/or aggregation-inhibiting substance.

5. Container (1) as claimed in one of claims 1 to 4, wherein the thixotropic substance (4) is selected from a material from a group comprising silicic acid, bentonite, hectorite, kaolin, alginate and/or a mixture thereof.

6. Container (1) as claimed in one of claims 1 to 5, wherein the separation medium (5) is an aqueous solution of at least one polymer, in particular with a silicate base, and/or a high-molecular carbohydrate, in particular saccharide, diatrizoate, e.g. Percoll®, Ficoll® or media with similar separating properties.

7. Container (1) as claimed in one of claims 1 to 6, wherein the separation medium (5) and/or the thixotropic substance (4) contains one or more dyes.

8. Container (1) as claimed in one of claims 1 to 7, wherein a porous barrier (6) is provided.

9. Container (1) as claimed in claim 8, wherein the porous barrier (6) is provided in the form of a membrane, flap, frit, sieve and/or filter.

10. Container (1) as claimed in claim 8 or 9, wherein the porous barrier (6) is displaceable.

11. Container (1) as claimed in one of claims 8 to 10, wherein the porous barrier (6) has a thickness selected from a range with an upper limit of 15 mm, preferably 10 mm, in particular 5 mm, and a lower limit of 0.1 mm, preferably 1 mm, in particular 2 mm.

12. Container (1) as claimed in one of claims 8 to 11, wherein the porous barrier (6) has a pore size selected from a range with an upper limit of 150 μm, preferably 100 μm, in particular 50 μm, and a lower limit of 1 μm, preferably 10 μm, in particular 20 μm.

13. Container (1) as claimed in one of claims 8 to 12, wherein the porous barrier (6) is made from a hydrophobic material and/or is provided with a hydrophobic coating.

14. Container (1) as claimed in one of claims 8 to 13, wherein the porous barrier (6) is bounded by an elastomer.

15. Container (1) as claimed in one of claims 8 to 14, wherein the porous barrier (6) is provided with an insertable closure element.

16. Container (1) as claimed in one of claims 8 to 15, wherein a projection is disposed on the internal face which holds the porous barrier (6) in its end position.

17. Use of the container (1) as claimed in one of claims 1 to 16 for separating tumour cells, in particular disseminated tumour cells (9), from a biological sample.

18. Method of identifying tumour cells, in particular disseminated tumour cells (9), comprising the following steps: (a) providing a container (1) as claimed in one of claims 1 to 16, (b) introducing the biological sample into the container (1), (c) centrifuging the container (1) in order to separate the biological sample into at least a bottom and top compartment (7, 8), (d) running molecular-biological, immunological and/or cellular tests with the tumour cells of the biological sample disposed in a compartment above the thixotropic substance and/or the porous barrier (6) after centrifugation.

19. Method as claimed in claim 19, wherein the biological sample is a body fluid from a group comprising blood and bone marrow, urine, saliva, lymph, exudate, transudate, spinal fluid, semen, or dispersed tissue and/or fluids from natural or non-natural body cavities.

20. Method as claimed in claim 18 or 19, wherein the tumour cells which can be identified include metastasing, in particular micro-metastasing tumours and/or neoplasms from a group of (1) solid tumours, comprising (i) epithelial tumours, such as lung carcinomas (lung carcinomas with small cells and not-small cells), gastrointestinal tumours (liver cell carcinoma, pancreatic carcinoma, oesophagus carcinoma, stomach cancer, intestinal cancer, colon-rectal carcinoma), breast cancer, liver and suprarenal tumours, cancer of the bladder and prostate carcinoma, and (ii) non-epithelial tumours, such as for example melanoma, neuroblastomas, brain tumours, rhabdomyosarcoma, leiomyosarcoma or teratocarcinoma, and (2) haematological tumours, such as for example T-cell lymphoblastomas, T-cell leukaemia, chronic myeloid leukaemia, acute lymphatic leukaemia, chronic lymphatic leukaemia, and/or lymphoma.

21. Method as claimed in one of claims 18 to 20, wherein the biological sample is diluted.

22. Method as claimed in one of claims 18 to 21, wherein the blood is taken in a coagulation-inhibiting substance.

23. Method as claimed in one of claims 18 to 22, wherein the biological sample has at least one aggregation-inhibiting substance added to it in order to prevent aggregation of thrombocytes on tumour cells.

24. Method as claimed in one of claims 18 to 23, wherein centrifugation is run with a g-number selected from a range with a lower limit of 500 g, preferably 800 g, in particular 1000 g, and an upper limit of 2500 g, preferably 2000 g, in particular 1500 g.

25. Method as claimed in one of claims 18 to 24, wherein the biological sample is centrifuged for a period with an upper limit of 60 minutes, preferably 45 minutes. in particular 30 minutes, and a lower limit of 5 minutes, preferably 10 minutes, in particular 20 minutes.

26. Method as claimed in one of claims 18 to 25, wherein the container is cooled, preferably to 4° C., in which case the preferred densities of (i) the thixotropic substance and optionally (ii) the separation medium are adjusted to this temperature.

27. Method as claimed in one of claims 18 to 26, wherein the container (1) is cooled after centrifugation and prior to removing the compartment containing the enriched tumour cells.

28. Method as claimed in one of claims 18 to 26, wherein the tumour cells are obtained from the compartment in and/or underneath the plasma compartment.

29. Method as claimed in one of claims 18 to 28, wherein the disseminated tumour cells (9) are removed from a top compartment (7) manually, semi-automatically and/or automatically.

30. Method as claimed in one of claims 18 to 29, wherein routine parameters relating to serology are determined from the uppermost compartment, which is plasma.

31. Method as claimed in one of claims 18 to 30, wherein for testing purposes, at least one method is selected from a group comprising immunocyto-chemical dying, PCR (Polymerase Chain Reaction), RT-PCR (Reverse Transcriptase-Polymerase Chain Reaction), cell culture, FISH (Fluorescence in-situ hybridisation) and/or FACS (Fluorescence activated cell sorter).

32. Arrangement of compartments of differing specific density of a biological sample and at least one separating medium for separating tumour cells, in particular disseminated tumour cells (9), in a container (1) with a closed end and an end which can be opened (2, 3), wherein initially in the region of the closed end (2), there is a separation medium (5) in the form of a density gradient with a specific density selected from a range with a lower limit of 1.060 g/cm³, preferably 1.065 g/cm³, in particular 1.070 g/cm³, and an upper limit of 1.085 g/cm³, preferably 1.080 g/cm³, in particular 1.075 g/cm³, and then optionally a thixotropic substance (4) with a specific density selected from a range with a lower limit of 1.055 g/cm, preferably 1.057 g/cm³ in particular 1.060 g/cm³, and an upper limit of 1.070 g/cm³, preferably 1.069 g/cm³, in particular 1.065 g/cm³, and in the region of the end (3) which can be opened, a space is disposed with a sufficient volume to accommodate the biological sample, and after centrifugation, (i) starting with the compartment in the region of the bottom end (2) of the container (1) are erythrocytes, then (ii) a compartment of leukocytes, lymphocytes, monocytes and optionally (iii) a part of the separation medium (5), in turn followed by (iv) the thixotropic substance (4), then (v) a compartment of diluted separation medium (5), followed by (vi) a compartment of plasma with thrombocytes and tumour cells, in particular disseminated tumour cells (9), and optionally (vii) a space is provided.

33. Test kit, wherein it contains a container (1) as claimed in one of claims 1 to 16.

34. Test kit as claimed in claim 33, wherein it contains at least one container (1) with a washing buffer, optionally in concentrated format.

35. Test kit as claimed in claim 33 or 34, wherein it contains other sample vessels.
